(19) United States
(12) Patent Application Publication
(10) Pub. No.: US 2026/0179735 A1
(43) Pub. Date: Jun. 25, 2026

(54) SYSTEM AND METHOD FOR PROVIDING HOSPITAL CUSTOMER RELATIONSHIP MANAGEMENT

(71) Applicant: FLYINGDOCTOR INC., Gyeonggi-do (KR)

(72) Inventor: Woo Jin Lee, Seoul (KR)

(73) Assignee: FLYINGDOCTOR INC., Gyeonggi-do (KR)

(21) Appl. No.: 19/128,912

(22) PCT Filed: Nov. 10, 2023
(86) PCT No.: PCT/KR2023/018038

§ 371 Date: Jul. 9, 2025

(30) Foreign Application Priority Data
Nov. 10, 2022 (KR) 10-2022-0149764
Nov. 9, 2023 (KR) 10-2023-0154525

(51) Int. Cl. G16H 10/20; G16H 10/60; G16H 40/20; G16H 40/63; G16H 40/67

(52) U.S. Cl. CPC G16H 10/20; G16H 10/60; G16H 40/20; G16H 40/63; G16H 40/67

(57) ABSTRACT

A method and a device for transmitting CRM content from a medical institution client assigned to a hospital or a doctor are provided. The medical institution client selects CRM content to be transmitted and, when an input of identification information of a recipient who is to receive the CRM content is received, performs CRM transmission. The CRM transmission is performed using a possible means regardless of whether a recipient has a membership and whether a service application has been installed, and is collectively managed in the future.

### TECHNICAL FIELD

The description below relates to a system and a method for providing customer relationship management (CRM).

### BACKGROUND ART

Customer relationship management (CRM) is an activity that manages communication with customers and conducts customized marketing. Nowadays, a hospital clinic (hereinafter, referred to as a ‘hospital’) also provides information to patients who are their customers through a text message or a social network service (SNS) and collects and processes inquiries, requests, and claims from the customers, thereby performing CRM for managing the patients. Such CRM in hospitals is also used as a management tool to increase patient satisfaction by actively communicating with patients and ultimately increase visit and revisit rates of the patients.

Such CRM in hospitals is a method in which hospital staff calls, sends emails, or sends a text message to patients at the timepoint of need, a method of texting a short message service (SMS)/a multimedia messaging service (MMS) text message in bulk by requesting an external service, or an app-based method of sending a message via a push message or instant messaging (such as KakaoTalk), and most of them are only one-way and one-time messages.

Meanwhile, telemedicine uses telecommunication and information technology to provide clinical healthcare from a long distance. Telemedicine removes a distance barrier and makes a medical service easily accessible. In many cases, telemedicine is used in a provincial area where it is difficult to continuously receive medical care due to distance. Telemedicine is also used to manage critically ill patients or save lives in emergency situations. Telemedicine is permitted under limited conditions for infectious disease prevention, and recently, in limited cases such as consultations of chronically ill patients or returning patients.

(Patent Document 1) Korean Patent Publication No. 2019-0135692 (published on Dec. 9, 2019) discloses a hospital customer relationship management (CRM) system that extracts and processes information that is necessary for patients by using medical electronic medical record (EMR) data, transmits the information to customers in the form of a message, or provides the information to a hospital terminal.

(Patent Document 2) Korean Patent Publication No. 10-1250462 (published on Apr. 8, 2013) discloses a hospital CRM system that enables hospitals to provide a counseling service to existing patients based on an EMR of the existing patients and actively attracts new patient customers by discovering marketing targets, that is, potential medical service users.

(Patent Document 3) Korean Patent Publication No. 10-0844543 (published on Jul. 8, 2008) discloses a method and device for implementing a web-based remote diagnosis.

(Patent Document 4) Korean Patent Publication No. 10-0366816 (published on Jan. 9, 2003) discloses a device and method for telemedicine.

### DISCLOSURE OF THE INVENTION

### Technical Goals

By connecting a hospital to patients, a customer relationship management (CRM) system, which serves as a mutual communication window for the patients with the hospital and as a system for the hospital to actively and proactively manage the patients and potential patients, may be provided.

A hospital-optimized CRM service, which provides appropriate information and alarms before a consultation, provides patients with sufficient necessary information after a consultation, and continues to care for patients, may be provided.

In addition, the flow of messages, which is the basis of CRM, may be continuously and consecutively integrated and managed. Furthermore, the flow of CRM messages through heterogeneous means may be managed to be integrated immediately or posthumously. CRM messages sent by a hospital client to a certain patient (or conversely, messages sent by a patient to a hospital client) may be integrated and managed regardless of whether an application (APP) is installed. Depending on the situational needs, the flow of CRM information transmitted by heterogeneous channels through different paths may also be integrated and managed immediately or posthumously.

Furthermore, the content that may not be conveyed through a general message (a short message service (SMS) or a multimedia messaging service (MMS)) may include rich and understandable content together with a picture/a video, contributing to enhancing patients'satisfaction with the hospital and the completeness of consultation throughout the entire period.

### Technical Solutions

### System Configuration and CRM Transmission

A computer-implemented method including receiving, from a medical institution client assigned to a hospital or a doctor, a selection of customer relationship management (CRM) content to be transmitted and an input of identification information of a recipient who receives the CRM content and transmitting the CRM content to a terminal of the recipient by a first means, in response to the input, may be provided. The computer may be an electronic device including at least one processor, a communication interface, and a storage. The computer may be communicatively connected to a patient terminal and a medical institution (hospital) terminal and configured to provide a service of transmitting, to the patient terminal, the CRM content from a client of the medical institution terminal.

### Integration of CRM Transmission Content Through Heterogeneous Means

According to an embodiment, when it is determined that the recipient has not subscribed to a service or that an application (APP) of the service is not installed on the terminal of the recipient, using the identification information of the recipient, the CRM content may be transmitted to the terminal of the recipient by a second means. In addition, the transmission by the second means may be stored in a buffer.

According to an embodiment, when the recipient subscribes to the service and the APP of the service is installed on the terminal of the recipient, the buffered CRM content may be integrated and managed by being merged into a CRM record in the APP of the service. In this case, a previous buffer may be flushed.

### Linkage to or Integration with Hospital EMR

For example, but without being limited thereto, a medical institution (hospital) client may be linked to or at least partially integrated with electronic medical record (EMR) (electronic chart) software used in the hospital. In an embodiment, at least a portion of the identification information of the recipient for the CRM to be transmitted may be extracted from the EMR software. The extraction may be performed automatically from an EMR consultation card opened in a corresponding consultation at the end of the consultation. In other cases, the extraction may include searching for and selecting by the patient's name, phone number (some of phone numbers), disease name, and the like. Furthermore, a target who receives the CRM does not necessarily have to be one person and may be people depending on embodiments.

### Examples of CRM Information

The CRM information provided by the hospital to the patient may vary. According to an embodiment, the CRM content may include information guiding content and the date and time of a consultation of at least one of the timepoints before, on, and after the scheduled consultation date.

According to another embodiment, the CRM content may include a survey on consultation satisfaction after a consultation.

According to another embodiment, the CRM content may include a digital prescription selected by the doctor, in which the digital prescription may include at least one of a visual analogue scale (VAS)-recording module, a body temperature measurement-recording module, a prescribed medication guidance module, a blood pressure-recording module, and a blood sugar-recording module.

In addition, according to embodiments, the CRM may include guidance on management after a consultation or procedure and a mobiDoc Feed, which is a professional column of the doctor.

### Guidance on Teleconsultation Included in CRM

According to an embodiment, according to guidance permitted by law, guidance on teleconsultation and/or hyperlinks to apply the teleconsultation may be optionally inserted into the CRM to be transmitted at the date and time when legal conditions are satisfied.

For example, when the record of the consultation by the recipient at the hospital is identified within a predetermined period by performing a reverse operation from the time of transmission so that the teleconsultation for a follow-up consultation is permitted when a corresponding CRM message is sent, an application for telemedicine (a link to apply for telemedicine) provided by the medical institution client may be provided by being included in the CRM content.

Furthermore, in the case of D+Day CRMs that are provided as follow-ups on the dates after a consultation or procedure, not the CRMs that are provided in advance in the form of D-Day for appointment dates, guidance on teleconsultation and application links may be provided even when the follow-up consultation conditions are satisfied. For example, among CRM content configurations, such as one day after D+1 and three days after D+3 the consultation or procedure date D, such guidance on the teleconsultation and the application link may be excluded at the timing when the follow-up consultation is not permitted (such as more than 30 days from the last face-to-face consultation date).

### Effects of the Invention

According to embodiments, a hospital may be actively connected to patients, and the patients may be managed by staying connected to the hospital. The patients may be satisfied with the mutual communication with the hospital as customers, and the hospital may manage the customers, thereby increasing the patient safety, patient satisfaction with the hospital, and revisit rate.

A hospital-optimized customer relationship management (CRM) service may be provided by continuously caring for the patients in advance, on the day, and posthumously, based on the patient's consultation and procedure schedule.

Pre-configured CRMs for each disease of patients and/or each procedure to be performed on the patients may be prepared and transmitted, the medical professionals/hospitals providing the CRMs may feel comfortable, and the patients may also receive timely information, increasing convenience and satisfaction with the consultation.

The various pieces of information and functions included in the CRM may contribute to increasing the patient's satisfaction with the hospital and the completeness of consultation throughout the entire period. In particular, even the content that may not be conveyed through a general message (a short message service (SMS) or a multimedia messaging service (MMS)) may include rich and understandable content together with a picture/a video, contributing to enhancing patients'satisfaction with the hospital and the completeness of consultation throughout the entire period.

Furthermore, the flow of messages, which is the basis of CRM, may be continuously and consecutively integrated and managed, so patients who have not yet signed up for membership or patients who have not installed an application (APP) may be integrated and managed with the previous CRM when the patients install the APP as members later.

In addition, various effects are provided by the embodiments and will be further described throughout the specification along with descriptions of the embodiments.

### BRIEF DESCRIPTION OF DRAWINGS

FIG. 1 is a diagram illustrating a connection between a device for providing customer relationship management (CRM) and a patient terminal, and between the device for providing the CRM and a doctor terminal.

FIG. 2 illustrates a device for providing CRM according to an embodiment.

FIG. 3 is a flowchart illustrating a method of transmitting and processing a CRM message, according to an embodiment.

FIG. 4 illustrates consultation guidance CRM being transmitted according to an embodiment.

FIGS. 5 to 10 are reference screens illustrating the types of CRM content and respective characteristics.

FIG. 11 illustrates CRM transmitted to a patient terminal, according to an embodiment.

FIG. 12 illustrates a telemedicine service being provided according to an embodiment.

### BEST MODE FOR CARRYING OUT THE INVENTION

Hereinafter, the embodiments will be described in detail with reference to the accompanying drawings. The scope of the right, however, should not be construed as limited to the embodiments set forth herein. In the drawings, like reference numerals are used for like elements.

The terms used herein are selected from terms generally understood by those skilled in the related art, but may have different meanings according to technical developments and/or changes, practices, and preferences of an engineer. Accordingly, the terms used herein should not be construed as limiting the technical spirit, and should be construed as illustrative terms to describe embodiments.

In addition, in a specific case, most appropriate terms are arbitrarily selected by the applicant. In this instance, the meanings of the arbitrarily used terms will be clearly explained in the corresponding description. Hence, the terms should be understood not by the simple names of the terms but by the meanings of the terms and the following overall description of this specification.

A device for providing customer relationship management (CRM) according to an embodiment may be an electronic device. The electronic device may include all types of system-on-chip hardware and/or a general-purpose computing device implemented by application (app) software if the device provides a service connection and implementation.

For example, the electronic device may include at least one of a smartphone, a tablet personal computer (PC), a mobile phone, a video phone, an e-book reader, a desktop PC, a laptop PC, a netbook computer, a personal digital assistant (PDA), a portable multimedia player (PMP), an MP3 player, a mobile medical device, a camera, or a wearable device (e.g., a head-mounted-device (HMD) such as electronic glasses, an electronic garment, an electronic bracelet, an electronic necklace, an electronic appcessory, an electronic tattoo, or a smartwatch).

According to an additional embodiment, the electronic device may include at least one of various medical devices (e.g., magnetic resonance angiography (MRA), magnetic resonance imaging (MRI), computed tomography (CT), an imaging device, an ultrasound device, etc.), a navigation device, a global positioning system (GPS) receiver, an event data recorder (EDR), a flight data recorder (FDR), a vehicle infotainment device, an electronic device for vessels (e.g., a marine navigation system and a gyro compass, etc.), avionics, a security device, a vehicle head unit, an industrial or household robot, an automatic teller's machine (ATM) of a financial institution, or a point of sales (POS) of a hospital.

In addition, an electronic terminal may include at least one of a portion of a building, a structure, or intelligent furniture for connecting telemedicine services, an electronic board, an electronic signature receiving device, a projector, or various measuring instruments (e.g., water, electricity, gas, or radio wave measuring instruments, etc.). The electronic device according to various embodiments of the present disclosure may be one of the various devices described above or a combination thereof. In addition, the electronic device according to various embodiments of the present disclosure may be a flexible device. It is obvious to one of ordinary skill in the art that the type of the electronic device may vary and the type and main purpose (other than the function of providing telemedicine services) of the electronic device are not limited to the descriptions provided above. Hereinafter, detailed descriptions of embodiments are provided with reference to the drawings.

### CRM Service Provided by mobiCoc™ According to Embodiments

The mobiDoc™ service may provide CRM. mobiDoc may satisfy the customized needs of patients but may aim for a hospital-centered service with a different perspective from an existing service. As patients search for a doctor who may cure their diseases, a doctor/a hospital may also try to attract and manage patients suitable for their hospital in various ways.

For example, a hospital and a doctor may send text messages (consultation guidance, closure guidance, hospital event/promotion guidance, appointment guidance, etc.) to get patients to revisit, send a message through a social networking service (SNS) such as KakaoTalk, operate a website introducing the hospital, and recently, even with time investment, operate blogs and YouTube channels. However, the cost of text messages and promotional services is high, and there are no services optimized for a doctor and a hospital.

mobiDoc may provide advantages in this regard. When using the CRM function provided by mobiDoc, hospital clients (i.e., clients signed in and used by a doctor and clients signed in and used by hospital staff) may collaborate with each other and complement their roles. Accordingly, if a doctor desires to make the next appointment and provide special care for a patient immediately after finishing a consultation, the doctor may create or edit CRM and apply the CRM to the patient, and the hospital staff may also set the CRM in a time-efficient manner. Accordingly, the delay in consultation by medical personnel may be reduced, and the hospital may operate smoothly even in a hospital with insufficient staff compared to the work or when there is a temporary shortage of staff due to a surge of patients. In addition, compared to transmitting CRM in bulk via a short message service (SMS) or multimedia messaging service (MMS) by using a bulk text message-sending agent service, it may be possible to precisely customize for customers or hospitals, actively cope with changes in situations, and increase competitiveness in cost.

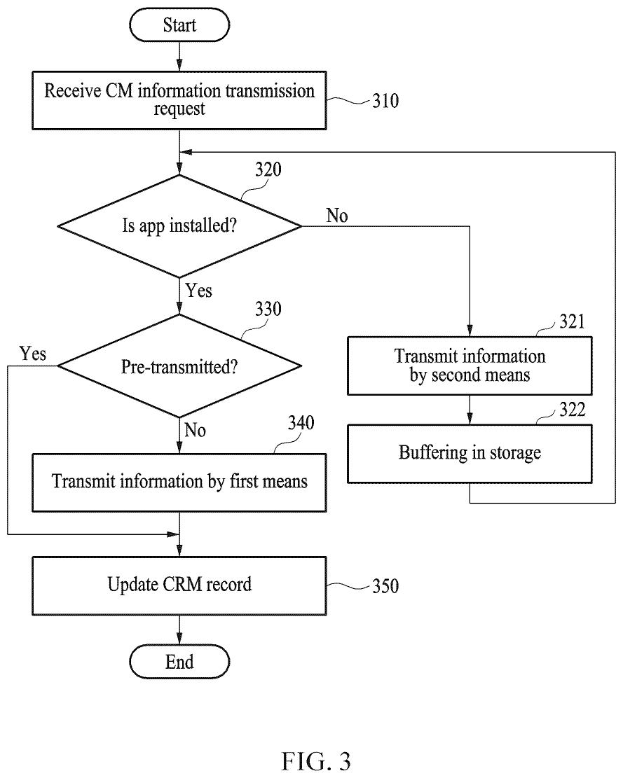

Additionally, the CRM of mobiDoc may include an appointment creation notification, a notification before the appointment date, a notification on the day of the appointment, guidance on an examination, guidance on a direction to the hospital, guidance on doctor's instructions, a digital prescription after a consultation, digital medicine, or a record/guide for monitoring consultation progress, guidance on care after a consultation or procedure, a mobiDoc Feed that is a professional column of a doctor, guidance on a teleconsultation application, an application link, and the like, which are transmitted to the patient. The types of CRM content and the corresponding process are described in more detail below with reference to various examples of FIGS. 3 to 10.

### System and Service Configuration

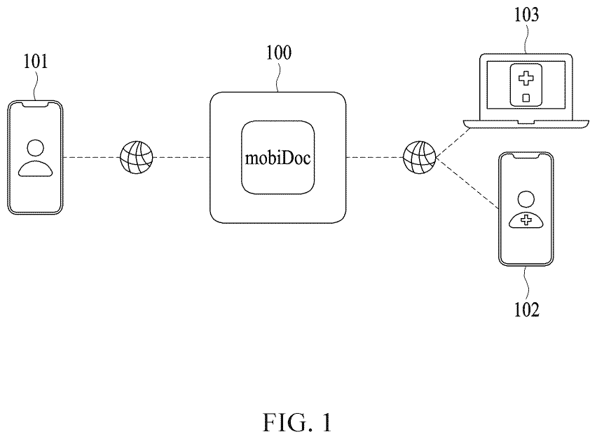

FIG. 1 is a diagram illustrating a connection between a device 100 for providing CRM and a patient terminal 101, and between the device 100 and terminals 102 and 103 belonging to the medical institution.

In the overview diagram, the device 100 may be implemented by a server-class computer, a workstation, a desktop computer, a laptop computer, a mobile electronic device, or the like and may also be implemented by a plurality of devices among the devices described above.

The device 100 may be connected to the patient terminal 101 through a network and may also be connected to a doctor terminal 102 and a hospital terminal 103. Each of the patient terminal 101, the doctor terminal 102, and the hospital terminal 103 may also correspond to a desktop computer, a laptop computer, or a mobile electronic device such as a smartphone or a tablet PC. Each of the patient terminal 101, the doctor terminal 102, and the hospital terminal 103 may have a software means, which is referred to as a ‘client’ for convenience.

A patient client may be executed in the patient terminal 101. A doctor client may be executed in the doctor terminal 102, and a hospital client may be executed in the hospital terminal 103. The doctor client may correspond to a counterpart of the patient client and may be installed at the hospital. There may also be an online cloud service in the form of Software as a Service (Saas). In this case, the doctor client may indicate a service form in which a cloud service is provided when accessed from an account of a doctor and/or hospital staff. In addition, the hospital client may be a means by which administrative staff or nurses of the hospital receive a consultation application, not medical personnel (doctors) who perform the consultation, and perform consultation assistance tasks such as receiving hospital expenses.

The doctor client used by a doctor and the hospital client 103 used by hospital staff, other than the doctor, may be provided separately. However, according to an embodiment, the doctor client and the hospital client may be integrated into one, and all functions may be activated when the sign-in account belongs to a user who is a doctor with a medical license, and some functions may be deactivated when the sign-in account belongs to staff, not a doctor. Some functions to be deactivated may be functions that only the doctor should perform, such as ‘start teleconsultation’ or the like. Since the time point when the consultation of the next patient is to be started may be most accurately and appropriately selected by the doctor, the ‘start a consultation’ function that initiates video access with the patient client who is waiting in the examining room may be implemented as a function that only the doctor may perform. However, this is an example, and hereinafter, it may be understood that the medical institution client collectively includes the doctor client and the hospital staff client, which are installed on the doctor terminal 102 and the hospital terminal 103, throughout the specification.

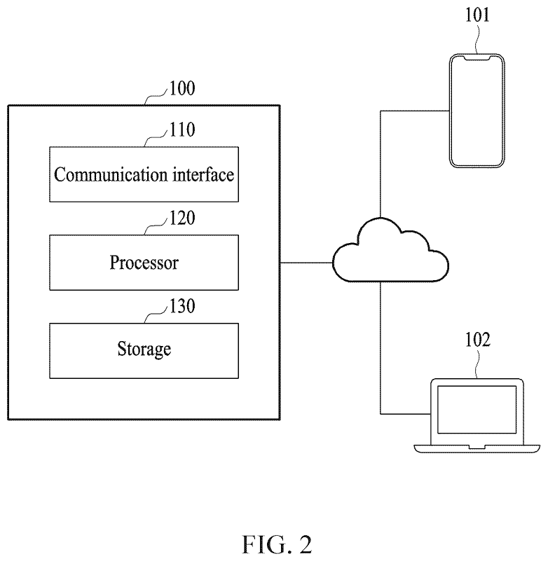

The client installed on the patient terminal 101, the doctor terminal 102, and the hospital terminal 103 may be an original app software to be installed, a service provision through a page accessed through a web-based browser, or a hybrid app. having both app and web characteristics. Furthermore, the client may be a cloud-based service provided in the form of SaaS. Since such an implementation is feasible for those of ordinary skill in the art, a further detailed description is omitted, and although the description of the embodiments mentions one example, the description does not exclude another implementation. The implementation and operation of the device 100 are described with reference to FIG. 2.

### System Configuration and CRM Transmission

FIG. 2 illustrates the device 100 for providing the CRM according to an embodiment. The device may be an electronic device including at least one processor 120, a communication interface 110, and a storage 130. The device may be communicatively connected to the patient terminal 101 and the medical institution (hospital) terminal 102 and may provide a service of transmitting CRM content from a client of the medical institution terminal to the patient terminal.

According to an embodiment, a method of providing CRM provided by the device 100 may include receiving, from a medical institution client assigned to a hospital or a doctor, a selection of CRM content to be transmitted and an input of identification information of a recipient who receives the CRM content and transmitting the CRM content to a terminal of the recipient, in response to the input.

According to an embodiment, the medical institution client may provide a plurality of preset CRM templates as selectable options. The CRM content to be transmitted through a user interface from the medical institution client may then be selected from among the options. Depending on the patients and the diseases, the selected CRM may be appropriately modified and transmitted by the doctor who performs a consultation or hospital staff.

### CRM Transmission Process and Integrated Management

FIG. 3 is a flowchart illustrating a CRM transmission process according to an embodiment.

In operation 310, the device 100 for providing a service may receive a CRM information transmission request. The request may be from a hospital client of the hospital terminal 102.

The request may include identification information of a recipient who is a target of the CRM information transmission, and in operation 320, the device may check whether a mobiDoc™ app is installed on the patient terminal 101. For example, but without being limited thereto, this may be possible by checking the demographic information, customer number, phone number, or the like of a patient in a database (DB) of the storage 130.

When the app is installed, in operation 340, a first means (e.g., transmitting a push message through the mobiDoc app) may be performed, and in operation 350, a CRM transmission record for a corresponding patient may be updated to the storage 130.

In operation 320, when it is determined that the app is not installed on the patient terminal, the push transmission through the app may not be performed in this case, so in operation 321, a second means (e.g., transmitting CRM through text messages or a messaging service such as KakaoTalk) may be performed. In operation 322, the CRM transmission through the second means may then be stored in the storage 130. Here, the storing in operation 322 may be integrated and managed as a CRM record in the app after the app is installed later, and until then, the CRM may be temporarily transmitted to a patient who is not a member or does not install the app, but the content and log may be temporarily recorded, so the expression, buffering, may be used.

When the information is buffered and the patient installs the app later, in operation 350, the past CRM messages that are buffered in operation 322 before the installation timepoint may also be integrated and updated as the CRM record. The buffered CRM transmission records in the storage at that time may be flushed.

Even if the patient does not install the mobiDoc app or is not a member, the patient may not be excluded from the CRM transmission target and access the mobiDoc CRM through a different path, other than the mobiDoc app. Accordingly, when the patient receives such a request through a text message for now, (apart from the process of recommending the installation of the mobiDoc™ app) the patient may access the mobiDoc service based on the web through a hyperlink, thereby checking the content or answering questions. Through this process, non-members or customers who do not install the app may also be induced to sign up for mobiDoc and install the app in the future, and after installation, the CRM activities that were previously performed when the patient was a non-member or before installing the app may all be confirmed without a sense of difference.

According to embodiments, the flow of messages, which is the basis of CRM, may be continuously and consecutively integrated and managed. Since the flow of CRM messages through heterologous means may be integrated immediately or posthumously, even when the CRM messages are distributed heterogeneously through different paths depending on whether the app is installed or situational needs, the flow of CRM information may be integrated and managed immediately or posthumously. Various flow paths of CRM information, such as an app-based, web-based, wired/wireless call, or message-based information provision (including both sending messages and providing necessary information) may coexist but may be integrated and managed, and from the hospital's perspective, longitudinal following and management of patient CRM may be possible. From the patient's perspective, even when the patient deletes a certain app and resets the app later, or even when the patient receives management from the hospital without installing the app and then later installs the app, it may be convenient because the contents of continuous communication with one hospital are integrated and managed.

### Examples of CRM Information

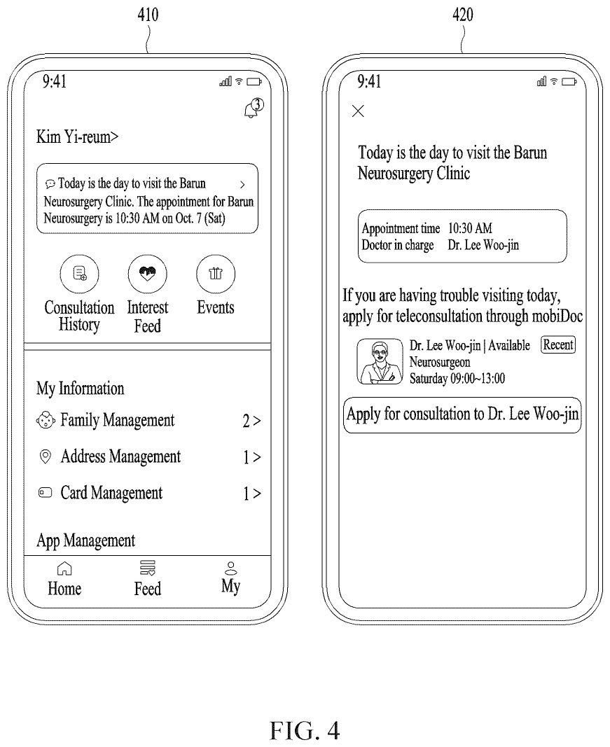

The CRM information provided by the hospital to the patient may vary. FIGS. 4 to 10 are various examples of CRM information and examples of screen configurations illustrating respective characteristics. A detailed description is provided with reference to the drawings.

### Consultation Day Guidance (D-Day Guidance)

FIG. 4 illustrates CRM being transmitted according to an embodiment.

For example, in the case of an in-patient, guidance on the next visit schedule may be provided through CRM after a consultation is finished. In the case of a new patient, guidance on the date and time of the first examination may be provided. An alarm that informs of the scheduled consultation date (or vaccination date) may be displayed on a screen 410 so that busy workers, elderly people with poor memory, and parents busy with childcare do not miss the next consultation date or vaccination date. In addition, a CRM message from a patient who requests an appointment change may be transmitted to a hospital by checking if an appointment change is required, in addition to simply informing the appointment date.

In the shown example, CRM may be transmitted on the day of consultation D-0, but each CRM may also be transmitted on D-7, D-3, D-1, or the like. In addition to information indicating that today is a day or three days before the consultation, the patient scheduled for an examination may be informed of medication to stop taking and the length of time to fast.

Additionally, in medically appropriate cases, such as in the case of a re-consultation in which the patient's progress is examined and whether to change a prescription is checked, or in the case in which main consultation content is to inform the patient who visited the hospital in advance of the examination results and issue a prescription, the visiting appointment may be changed to a remote video consultation (mobiDoc Clinic) according to the choice of the doctor or patient, as shown in a screen 420. In this case, the hospital may first transmit CRM information inquiring the patient whether to change the consultation to the remote video consultation because the consultation to be conducted today may be performed through the video consultation, and conversely, the patient who is visiting the hospital may also transmit information requesting the hospital to change the consultation to the remote video consultation (mobiDoc Clinic).

### Survey on Satisfaction with Consultation

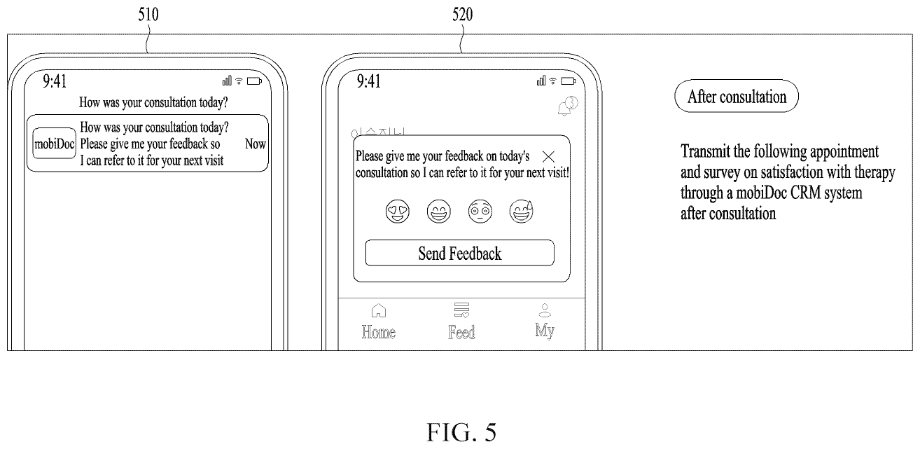

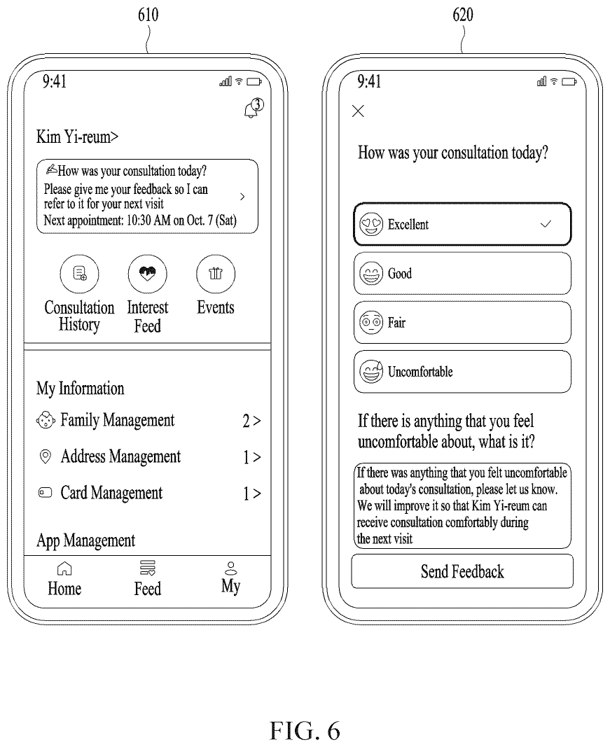

Another example of the CRM information provided by the hospital to the patient may be a survey on satisfaction with consultation. FIGS. 5 and 6 illustrate embodiments in this regard.

When the consultation is finished, a request for feedback on satisfaction with consultation may be transmitted to the patient client, as shown in the example of a screen 510, and when the patient responds to this, a survey screen 520 may be provided and the survey may begin. As shown in the screen 510 of FIG. 5, a push alarm may induce a survey response, but when the patient executes the app first, the survey response may be included in an app screen 610.

As shown in a screen 620, the survey may include objective and/or subjective questions about the doctor who performs the consultation, the physical therapist, the hospital staff, cost, and consultation time, as well as the overall satisfaction. The CRM of the survey on satisfaction with consultation may be an important reference for hospital management from the perspective of hospital managers. The patient may also experience such CRM and feel satisfied that ‘the hospital is re-checking the patient's satisfaction.’ Compared to the experience of receiving promotional text-oriented CRMs from hospitals in the past, the CRM service according to an embodiment may provide much more immediate and improved customer experience.

The transmission of the CRM information requesting feedback from the patient may also be performed according to the process described above with reference to FIG. 3, depending on whether the patient installs the mobiDoc app. When the patient has not yet installed the app that provides the CRM service, such as the mobiDoc app, even when the patient has not installed the mobiDoc app or is not a member, the patient may not be excluded from the survey on satisfaction with consultation and may be provided with an experience of responding to the mobiDoc CRM through a different route for now, other than the mobiDoc app, as described with reference to FIG. 3.

As described above, when the patient receives such a request through a text message for now (apart from the process of recommending installation of the mobiDoc™ app), the patient may access the mobiDoc service based on a web through a hyperlink and respond, which may then be stored and managed in the CRM DB for the patient. In addition, after the patient installs the mobiDoc™M app later, the patient may check the CRM information transmission history and content, which are previously transmitted from the app, all together through the process described above. Accordingly, non-members or customers who have not installed the app may also be encouraged to join mobiDoc and install the app in the future, and after installation, the CRM activities that were previously performed when the patients were non-members or before installing the app may all be checked without a sense of difference.

### mobiDoc Feed™ Content

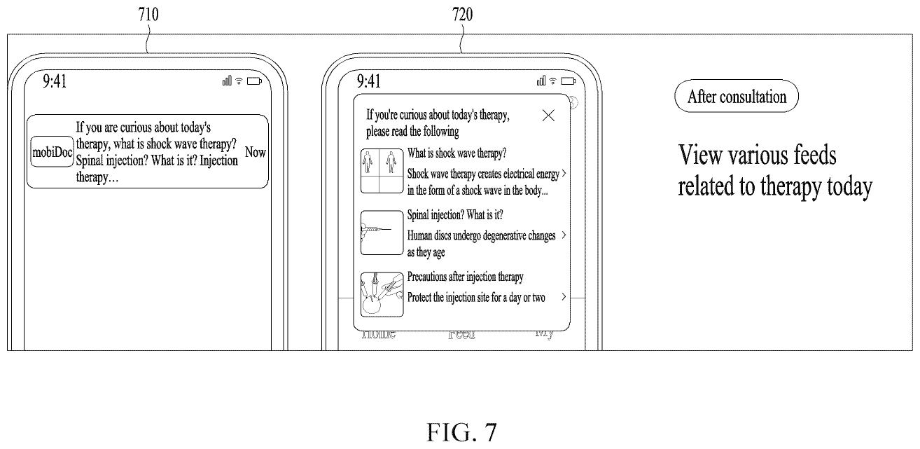

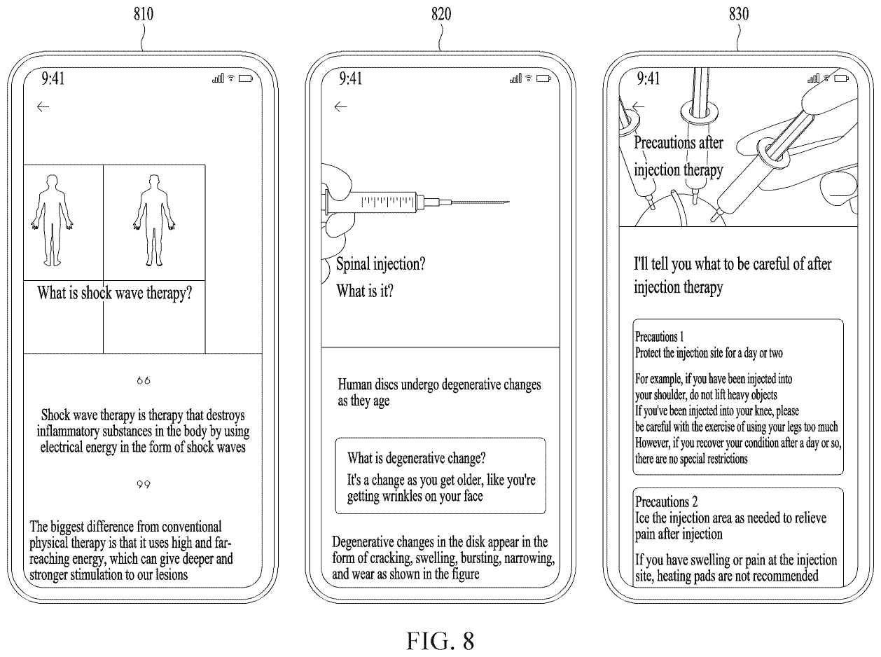

Another example of the CRM information provided by the hospital to the patient may be the mobiDoc Feed content provided through the mobiDoc™ app. FIGS. 7 and 8 illustrate examples in which a feed about a consultation conducted today is transmitted after the consultation.

According to an embodiment, a push alarm may be provided in the form of a screen 710. The patient who came to the hospital, received shock wave consultation, and returned home may wonder what role the shock wave consultation plays and why the shock wave consultation is helpful, even though the doctor prescribed the shock wave consultation and the patient received the shock wave consultation in the physical therapy room. In general medical sites, personnel, such as doctors and physical therapists, may explain the consultation, but each person has different explanation skills, and sometimes the explanation may not be faithful when busy. The patient may also quickly forget the explanation during a consultation or physical therapy (sometimes during a procedure) because the patient is absent-minded. Some patients may not be able to ask all the questions the patients have due to their personalities. mobiDoc may satisfy both the medical professional and the patients in this regard. mobiDoc may save the medical professional's time while providing sufficient information to the patient. By simply checking the feed on the details of the consultation transmitted by the hospital to the patient who returns home after finishing the consultation, the patient may be well informed of the consultation details without missing any information. A screen 720 shows, for example, a feed about the shock wave consultation the patient received today, a feed about the spinal injection, and a feed containing precautions for the injection received today.

For reference, the mobiDoc Feed may be a content-providing service that medical-licensed members provide to patient users who follow the medical-licensed members or their hospitals, through the doctor client or the separately provided mobiDoc web. For example, but without being limited thereto, the mobiDoc Feed may include the content provision that includes text, images, videos, etc., containing disease information, consultation trends, medical common sense, and health management. Doctor members may usually write such feeds about the diseases for which the doctor members are responsible, procedures, and consultation methods, and may choose and transmit necessary content while treating patients.

A screen 810 is an example in which a doctor who frequently performs a shock wave consultation writes a feed in advance about things that a patient may be curious about and transmits the feed to the patient who received the shock wave consultation today. A screen 820 is an example in which a doctor writes a feed about the injection and the disease and transmits the feed to a patient who received the spinal injection today. A screen 830 shows precautions after the injection consultation.

Furthermore, the feed may also include a link that directly provides a mobiDoc appointment to a doctor who writes a corresponding feed or a doctor or hospital recommended by the doctor for a corresponding disease. When a patient has had a procedure or consultation, such as physical therapy, injection therapy, or laser therapy, a means of providing additional information about the disease, a future consultation plan, an explanation of the consultation or procedure conducted today, precautions, and/or detailed information about prescribed medications may be customized and transmitted to the patient in the form of CRM information transmission under the name of the mobiDoc Feed.

The transmission of the customized provision of the mobiDoc Feed content as CRM may be performed in the form of in-app push and content viewing for the patient who has installed and is using the mobiDoc™ app. However, for the patient terminal that does not have the mobiDoc™ app installed, the mobiDoc Feed content may be provided for now by another contact method that is identified by patient information, and after the patient installs the mobiDoc™ app in the future, the mobiDoc Feed content may be updated in the existing CRM information exchange history so that continuous and consecutive CRM management may be performed without a sense of difference.

### Management of Patient After Consultation/procedure Through CRM (D+Day CRM)

Further from the embodiments with reference to FIGS. 7 and 8, according to another embodiment, D+Day CRM for tracking and managing a patient after a consultation or procedure for a certain period of time and for providing information may be provided, in addition to the day of consultation.

For example, when a patient had a procedure at the dermatologist, there may be cases in which follow-up care is necessary according to the schedule, such as what measures to take and what to be careful of one day after the procedure, what information should be provided to the patient three days after the procedure, making a contact with the hospital if there is an abnormality after checking one week after the procedure, or the like. Previously, most dermatologists provided printed instructions for such cases, which may cause various inconveniences. In addition to dermatological procedures, there may be these needs in most cases, such as several surgical procedures, including plastic surgery, pain intervention, cardiac intervention, and medical measures for certain symptoms. Even during the COVID-19 pandemic that has swept the world in recent years, there may be a need to provide systematic information to the patients and help the patients manage their health by providing daily guidance to infected patients, and mobiDoc CRM may play a role in this area in the future.

### Digital Prescription CRM Modules

Furthermore, another example of the CRM information provided by the hospital to the patient may include modules such as a digital prescription, digital medicine, and a survey on pain or disease status, which are provided through the mobiDoc app. The difference from the existing healthcare app that records weight, blood pressure, blood sugar, etc., may be that mobiDoc CRM is not a health record in the form of separate installation and execution by the patients themselves but is the execution of a function that is customized and prescribed directly by the “Doctor who treated me” by setting the period and content in the “My mobiDoc Account” in the mobiDoc platform. That is, in the extension of diagnosis and consultation, the ‘Doctor who treated me’ may directly execute a certain type of module through the doctor client 102 of mobiDoc, record something “from when, for how many days, and in what cycle”, and attach which survey the patient answers to the ‘My mobiDoc Account,’ thereby executing the module as prescribed through the patient terminal 101.

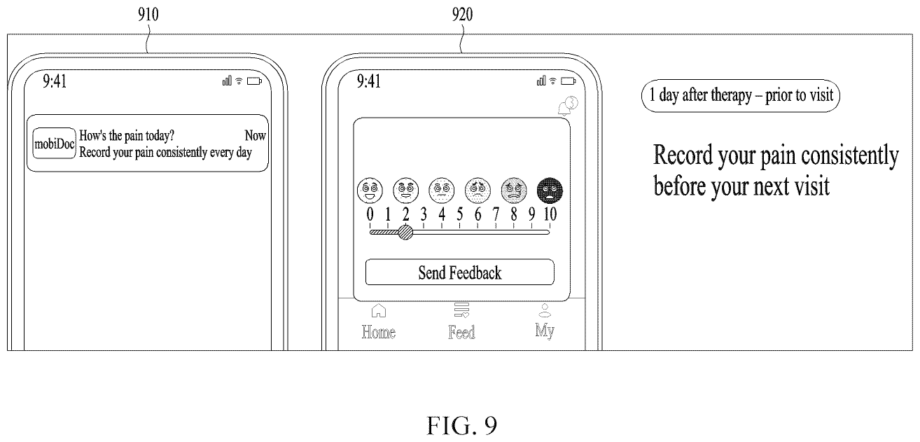

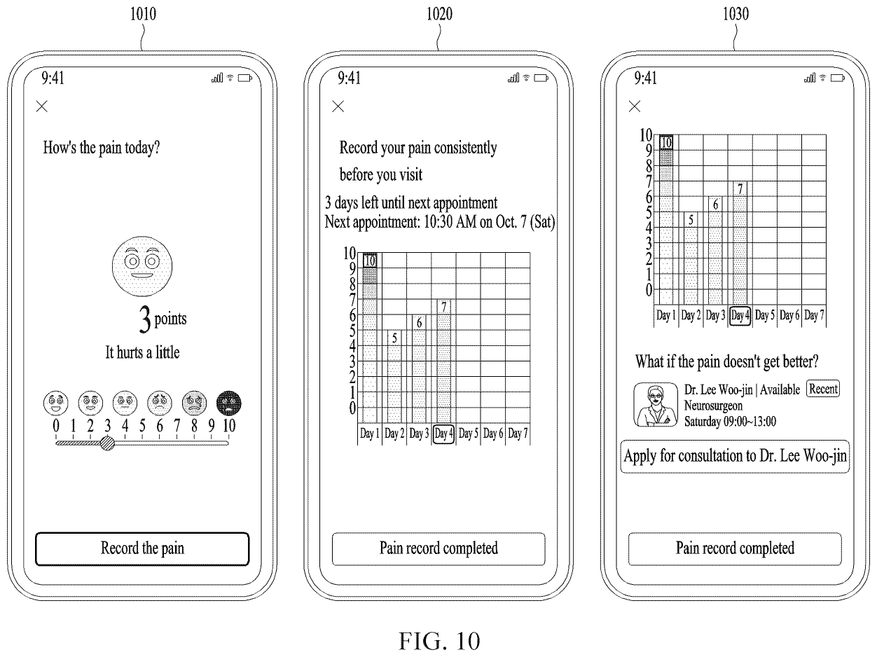

FIGS. 9 and 10 illustrate examples of screens in which a pain-recording module is prescribed and executed on the patient client 101, according to an embodiment. In orthopedics, neurosurgery, pain medicine clinics, etc., a digital prescription module for tracking the level of pain may be provided to the patient by CRM according to an embodiment of the present disclosure. The patient may not be under the control of the hospital and doctor from the time the patient finishes the consultation and returns home. In the case of a pain survey (e.g., visual analogue scale (VAS)), if a patient who has finished the consultation and continuously records their status and the hospital may monitor the records, which may contribute to the consultation and recovery of the patient.

An alarm 910 notifying to record pain may be provided at a predetermined cycle by the doctor, and when the patient responds to this, a VAS module 920 may be executed. The patient may record pain consistently as prescribed by the doctor who treated the patient by passively responding without having to take care of the pain recording on their own. Data according to the flow of time may be monitored by the hospital and/or utilized to discover abnormal situations due to data trends.

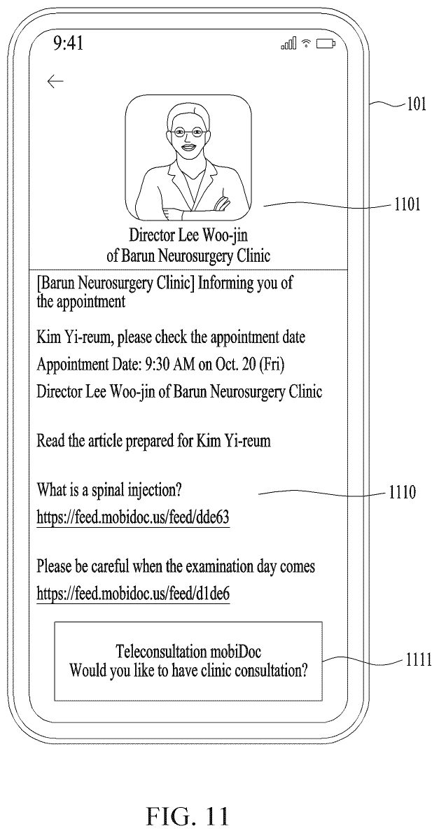

FIG. 11 is referred to together. The level of pain recorded by the patient in a VAS module 1010 that is executed according to the doctor's prescription may be managed as data according to the flow of time by the patient and the hospital and may also be visually displayed on a screen 1020. If the level of pain improvement is significantly lower than expected, depending on the hospital's choice, the hospital may inquire the patient whether the patient is taking the prescribed medication properly, whether the patient is following the precautions instructed by the doctor, etc., and if necessary, may induce the patient to bring forward the date of the next re-consultation appointment. Absolute values may also be considered, such as pain significantly exceeding what is generally expected of pain, but there may also be cases in which it is suspected that there are abnormal situations, such as the pain has become more severe than the previous measurement value or the pain is getting severe when the prescribed painkiller is still in time to take may also be found.

In such cases, according to an embodiment, a push alarm to remind the patient whether the patient is taking the prescribed medications properly and to continue taking the medication properly may be transmitted to the patient through CRM. According to another embodiment, with more active intervention, a CRM alarm may be transmitted to suggest the remote video consultation through the mobiDoc Clinic, as shown in a screen 1030, so the hospital may proactively interview the patient and change the prescribed medication or provide the patient with additional instructions of the doctor.

There may be many other examples of such digital prescription modules provided by mobiDoc CRM.

In another embodiment, in the case of pediatrics and adolescents, a digital prescription for recording body temperature may be modularized to manage the child's fever and be provided to a certain patient in the form of CRM for a certain period of time. From the perspective of caregivers who are raising children, when the caregivers respond to the CRM information communicating through the mobiDoc app after the consultation at the mobiDoc affiliated hospital, this may help with the consultation and prevent emergency situations by passively responding to the prescribed body temperature-recording paper for a certain period of time.

Furthermore, when patients who need to measure their blood sugar levels or blood pressure periodically respond to the recording paper prescribed to the patient in the mobiDoc™ app, the hospital may manage the patients and provide necessary suggestions.

### Ensuring Completeness of Consultation That Contributes to Patient Recovery

The modularized CRM may help a patient achieve emotional stability and contribute to improving the completeness of consultation. In addition, the fact that the hospital takes care of the patient even after the patient returns home after finishing the consultation, treats ‘out-patients like in-patients,’ and provides necessary suggestions to the patient by asking if the patient is okay first may be a new medical service presented by mobiDoc CRM according to embodiments. This customer experience (CX) of receiving medical care as if from a family member or close acquaintance may increase the patient's satisfaction and loyalty to the hospital, in addition to helping the patient recover and preventing dangerous situations. That is, from the hospital's perspective, this may contribute to increasing the re-consultation rate by increasing patients'satisfaction with the hospital. Furthermore, patients with mild symptoms should be discharged quickly due to a chronic lack of beds, such as in a high-level general hospital, but when the patients need care even after discharge, the patients may be managed through mobiDoc CRM. Accordingly, this may help to manage the patient's health while lowering the hospitalization rate.

### Example of Crm Screen Configuration

FIG. 11 illustrates CRM transmitted to a patient terminal, according to an embodiment.

Information on the hospital and doctor that transmit CRM from the patient terminal 101 may be displayed on an upper field 1101, while a CRM message may be provided in a lower field 1110. The layout of the upper field 1101, the lower field 1110, and a field 1111 illustrated in FIG. 11 is merely an example. Accordingly, layouts with other sizes may be possible. For example, unlike FIG. 11, it may also be possible to configure the layout like a message app interface of a smartphone or a direct message (DM) interface of an SNS.

The process of configuring the CRM message and content is as described above.

### Feed Content Included in CRM

As shown in FIG. 11, the CRM message may include information about diseases, health, and other specialized articles, which are transmitted to the patient by the doctor or hospital. The articles may be directly inserted but may also be presented in the form of hyperlinks, as shown. The hyperlink may connect a corresponding mobiDoc Feed article to the patient in the app and/or web. Since the mobiDoc Feed is written only by licensed doctors, the content may be verified. Accordingly, when the doctor who treated the patient inserts specialized column articles that are suitable for the patient's disease and body into CRM and transmits the CRM to the patient, the patient's attention and concentration on the CRM itself may become high. As described above, the patient may often perceive existing CRM messages in the form of SMS or MMS transmitted from hospitals as advertisements (ADs) and may not pay attention to the CRM messages. However, since the mobiDoc Feed is provided together in the CRM field 1110, as shown in FIG. 11, the patient may feel that the hospital and doctor who transmit the CRM message are truly treating the patient as a customer and taking care of the patient.

Accordingly, the positive function of CRM may be realized, allowing hospitals to manage and strengthen the relationships with their patients. The strengthening of the CRM function may be further enhanced when combined with the mobiDoc's teleconsultation, ‘mobiDoc Clinic.’

### Teleconsultation Guidance Included in CRM

Even when a patient makes the next consultation appointment (re-consultation appointment), there may be cases in which the rate of missed appointments is not low. There may be a variety of reasons why patients who made a re-consultation appointment miss their appointment, cancel their appointment, or do not visit (no-show) the hospital, but the reason may include not being sure whether a re-consultation is necessary and/or having difficulty visiting the hospital due to work or childcare. In the mobiDoc Clinic, the teleconsultation provided by mobiDoc™ may function as a nudge that allows these patients to easily access the re-consultation through CRM. It may also be expected that there is a positive effect for these patients to determine whether to make additional visits to the hospital while consulting again with the doctor who initially treated the patients about the disease's progress, current conditions, and side effects of prescribed medications, by applying for a brief teleconsultation.

Accordingly, according to an embodiment, guidance that a patient may apply for a mobiDoc teleconsultation and/or a hyperlink that a patient may apply for the teleconsultation may be provided as part of the CRM in the field 1111 of FIG. 11.

Furthermore, the references for allowing such teleconsultation may vary by country and period. Accordingly, according to an embodiment, guidance on teleconsultation and/or hyperlinks to apply for the teleconsultation may be optionally inserted into the CRM to be transmitted at the date and time when the legal conditions are satisfied according to the guidance allowed by the law.

For example, guidance and application links for teleconsultation may be provided in the field 1111 only when a re-consultation is within a pre-specified period, such as within 30 days when the teleconsultation is allowed by performing a reverse operation from the time the illustrated CRM is transmitted. For this purpose, the D-0 day (on the day of the previous appointment) information in the CRM according to the hospital's EMR and/or previous appointment information may be utilized and be compared with the time point when the CRM is transmitted later. That is, whether the CRM is transmitted 7 days after the previous consultation or 2 months later may be distinguished. When the reference for allowing the teleconsultation allows only a re-consultation for the follow-up consultation of the same disease at the same hospital within 30 days, the former (CRM transmitted after 7 days) may include the teleconsultation guidance and application link, but in the latter (CRM transmitted after 2 months), the teleconsultation guidance and application link may not be guided.

Furthermore, in the case of D+Day CRM, which performs follow-up management for a previous consultation or procedure, such teleconsultation guidance and application links may be provided only within a predetermined reference period (e.g., 30 days). For example, among the CRM content configurations, such as one day D+1 or three days D+3 after the consultation or procedure date D, such teleconsultation guidance and application links may be excluded at the timing when a re-consultation is not allowed (e.g., more than 30 days from the last face-to-face consultation date).

According to another embodiment, CRM asking whether to perform teleconsultation for a re-consultation before the described-above limit time point of the re-consultation allowance has passed may be transmitted. For example, teleconsultation guidance and links may be transmitted in the form of CRM to the patient who has had a first-time consultation or procedure one time after 21 days and one time after 28 days.

### mobiDoc Clinic (Teleconsultation) Starting Through CRM

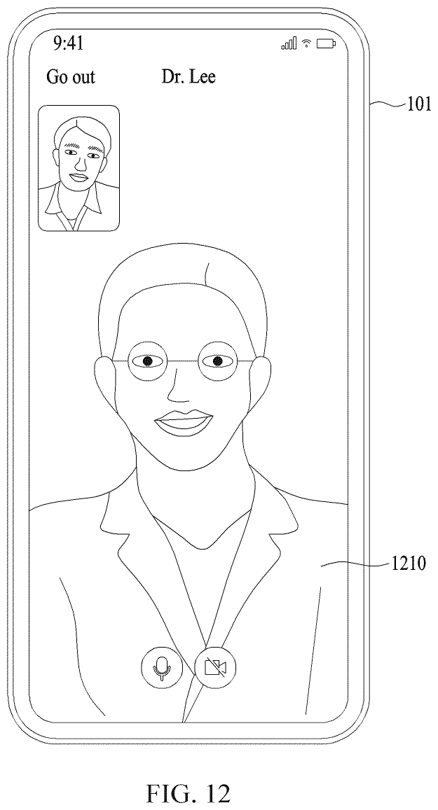

FIG. 12 shows the start and progress of teleconsultation by using a link in the field 1111.

Although not shown, a patient client may complete the consultation application and request access to an examining room. Here, the examining room may be a session where the patient waits for the doctor to start the consultation during the processing process of the mobiDoc Clinic, which is teleconsultation through mobiDoc. The examining room may play a role in ensuring that the patient waits for the doctor to start the consultation, rather than simply a state in which the teleconsultation application is completed.

After the consultation application is completed, the client of the patient's account may be admitted to a stage, called an examining room, based on the approval of the hospital and/or doctor. This may indicate that the service state of the patient client for the patient's account is changed from the consultation application process to the consultation waiting process, and the examining room may be a stage corresponding to a state in which a video call and/or a voice call may be started immediately when the doctor starts the consultation. This may be a user experience similar to that of an offline actual hospital, where a nurse guides the patient into the examining room, makes the patient wait in the place where the consultation is to be performed, and reports to the doctor that the patient is ready, and then the doctor comes in the place when the doctor is ready and starts the consultation with the patient. In university hospitals, there may be cases in which a doctor goes back and forth between two connected examining rooms for a consultation. The doctor may perform the consultation in one examining room, while a nurse may make the next patient enter and prepare the patient for the consultation in the other examining room. Here, information such as charts, necessary medical images, and test results may be prepared through the EMR or another means, allowing the busy doctor to avoid wasting time in the preparation process. While going back and forth between two examining rooms, the doctor may finish the consultation of a current patient in one examining room, perform necessary charting, then move to the other examining room where the next consultation is ready and the patient is waiting, and immediately start the next consultation. mobiDoc Clinic™ may provide these functions.

As a result, by reducing unnecessary waste of time, the doctor may substantially be faithful to the nature of consultation even when the doctor treats the same number of patients, patient discomfort may be reduced due to delays in consultation, and medical services may be provided to more patients.

The improvement in consultation efficiency that is achieved by making the patient wait and ensuring the possibility of immediate initiation of a consultation may be important because better service is provided to both medical professionals and patients. In most cases of existing telemedicine services, when a patient applies for online (remote) consultation, the doctor (or hospital) may attempt to access the patient. This access may be video calls, voice calls, etc. However, since teleconsultation is a situation in which people are connected online and not a situation in which people actually meet face to face, the patient may do other things while waiting for the teleconsultation with the doctor or leave the terminal and go somewhere else. In this case, the possibility of immediate consultation may not be guaranteed when the doctor (or hospital) desires to start a consultation.

The mobiDoc Clinic shown in FIG. 12 may check, through the patient client, whether the patient is actually ready for the consultation and whether the consultation is possible immediately when the doctor starts the consultation procedure. It may be checked that the patient is in the standby state, which is a state in which the patient enters the examining room, so the doctor client is aware of this situation.

If the check results on the patient client show that the patient is on a call, is not looking at the screen and is away from the room, or is performing other tasks using the terminal 101, and thus, the doctor cannot start the consultation when the doctor desires to and must wait, the doctor may be notified of this situation. In addition to notifying, the doctor may be reminded that ‘the patient may not be ready for the consultation’ and the doctor may be given the option to treat another patient first. It may be possible to create a nudge effect that allows the patient to be ready by giving the patient an alarm that “the consultation begins only when you wait for the consultation.”

With this consideration, the doctor may know which of the patients who apply for the consultation and enter the waiting room is ready for the start of a consultation and may select a well-prepared patient to initiate the consultation, thereby preventing the doctor and hospital from experiencing inefficiencies due to connection delays or connection failures. The patient who is not ready may be induced to prepare for the consultation (not turning off the app, not doing other tasks, and waiting while watching the screen of the examining room) by informing the patient terminal that the consultation begins only when prepared, from the hospital staff or telemedicine service provider. According to the embodiments, the possibility of immediate consultation for the patient by the doctor may increase.

FIG. 12 shows an appearance 1210 of the patient terminal when a consultation is started. The patient may be informed that teleconsultation is possible for a re-consultation and may receive the consultation without visiting the hospital in person. This case may be effective in consultation such as consulting with a doctor about the progress of the disease and controlling drug use without deliberately visiting the hospital when the need for re-consultation is unclear. Accordingly, teleconsultation may serve as a medium for a patient who is hesitating about whether to receive a re-consultation to continue the consultation with the doctor without giving up on the re-consultation. As a result, the gap in consultation that the patient may overlook may be reduced, and the situation in which health worsens may be prevented.

### Linkage or Integration With Hospital EMR

Although not shown, for example, but without being limited thereto, the medical institution (hospital) client may be linked to or at least partially integrated with EMR (electronic chart) software used in the hospital. In an embodiment, at least a portion of identification information of a recipient for CRM to be transmitted may be extracted from the EMR software. The extraction may be performed automatically from the EMR consultation card opened in the corresponding consultation at the end of the consultation. In other cases, the extraction may include searching for and selecting by the patient's name, phone number (some of phone numbers), disease name, and the like. Furthermore, a target to receive the CRM does not necessarily have to be one person and may be people depending on embodiments.

When the next appointment is created by the EMR during a consultation without going through mobiDoc or when the patient's appointment is created by another method, such as by calls, all or a selected portion of these items may be utilized for transmitting the appointment text message of mobiDoc™ CRM. Here, mobiDoc™ CRM may be API-linked with the EMR, and in this case, when a patient appointment is created in the EMR, an option to transmit an appointment notification text message may be provided through mobiDoc™ CRM. This linkage between mobiDoc™ CRM and the EMR may minimize the time of the doctor or hospital staff, which is used for CRM.

The units described herein may be implemented using a hardware component, a software component and/or a combination thereof. A processing device may be implemented using one or more general-purpose or special purpose computers, such as, for example, a processor, a controller and an arithmetic logic unit (ALU), a DSP, a microcomputer, an FPGA, a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter. The above-described devices may be configured to act as one or more software modules in order to perform the operations of the above-described embodiments, or vice versa.

A number of embodiments have been described above. Nevertheless, it should be understood that various modifications may be made to these embodiments. For example, suitable results may be achieved if the described techniques are performed in a different order, and/or if components in a described system, structure, device, or circuit are combined in a different manner, and/or replaced or supplemented by other components or their equivalents.

Therefore, other implementations, other embodiments, and equivalents to the claims are also within the scope of the following claims.

1. A computer-implemented method comprising:
receiving, from a medical institution client assigned to a hospital or a doctor, a digital prescription comprising recording pain at a predetermined interval for a predetermined period, based on at least one of a consultation, a procedure, and surgery received by a recipient;
selecting pieces of customer relationship management (CRM) content comprising a pain survey to be transmitted to the recipient at the predetermined interval for the predetermined period after the consultation, the procedure, or the surgery received by the recipient, based on the digital prescription;
transmitting the pieces of_CRM content to a terminal of the recipient at the predetermined interval for the predetermined period;
receiving, from the terminal of the recipient, pain scores representing the pain of the recipient recorded according to the predetermined interval for the predetermined period, based on responses that record the pain through the pain survey comprised in the pieces of CRM content;
generating pain data according to a flow of time based on the pain scores;
detecting an abnormal situation from the pain data based on at least one of a level of pain improvement shown in the pain data being less than or equal to a level of threshold improvement, an increase in a first pain score by a predetermined score difference or more compared to a second pain score measured immediately before the first pain score, or an increase in the pain scores recorded during a period of taking prescribed medication over time;
when the abnormal situation is detected from the pain data, selecting, from the medical institution client, additional CRM content comprising at least one of an inquiry about whether the recipient is taking prescribed medication, an inquiry about whether the recipient is complying with precautions, a suggestion for a re-consultation appointment, and a suggestion for adjusting a re-consultation appointment date; and
transmitting the additional CRM content to the terminal of the recipient.

2. The computer-implemented method of claim 1, wherein the transmitting of the pieces of CRM content further comprises:
when the recipient a subscriber to a CRM service and a CRM application for the CRM service is installed on the terminal of the recipient, transmitting at least one of the pieces of CRM content to the terminal of the recipient through the CRM application; and
when the recipient is not a subscriber to the CRM service or the CRM application is not installed on the terminal of the recipient, transmitting at least one of the pieces of CRM content to the terminal of the recipient through a different application from the CRM application.

3. (canceled)

4. The computer-implemented method of claim 1, wherein the transmitting of the pieces of CRM content comprises:
when the recipient is not a subscriber to a CRM service or a CRM application is not installed on the terminal of the recipient, transmitting at least one piece of CRM content among the pieces of CRM content to the recipient through a different application from the CRM application;
recording the at least one piece of CRM content a storage when transmitting the at least one piece of CRM content through the different application;
when the recipient subscribes to the CRM service and the CRM application is installed on the terminal of the recipient after transmitting the at least one piece of CRM content through the different application, obtaining, from the storage, the at least one piece of CRM content transmitted to the recipient before the CRM application is installed;
when the recipient subscribes to the CRM_service and the CRM application is installed on the terminal of the recipient, transmitting remaining CRM content among the pieces of CRM content to the terminal of the recipient through the CRM application; and
displaying together the at least one piece of CRM content transmitted to the recipient before the CRM application is installed and the remaining CRM content transmitted to the recipient after the CRM application is installed, through the CRM application installed on the terminal of the recipient.

5. The computer-implemented method of claim 1, wherein the medical institution client is linked to or integrated with electronic medical record (EMR) software used in the hospital,
wherein at least a portion of identification information of the recipient is extracted from the EMR software.

6. The computer-implemented method of claim 1, wherein the pieces of CRM content comprise information guiding content and a date and time of a consultation of at least one of timepoints before, on, and after a scheduled consultation date.

7. The computer-implemented method of claim 1, wherein the pieces of CRM content comprise a survey on consultation satisfaction after a consultation.

8. The computer-implemented method of claim 1, wherein the digital prescription
comprises at least one of a visual analogue scale (VAS)-recording module, a body temperature measurement-recording module, a prescribed medication guidance module, a blood pressure-recording module, and a blood sugar-recording module.

9. The computer-implemented method of claim 1, wherein the transmitting of the pieces of CRM content comprises:
determining whether the hospital permits teleconsultation of the recipient when transmitting each piece of CRM content;
adding a link to apply for the teleconsultation to corresponding CRM content when the teleconsultation is permitted, when transmitting each piece of CRM content; and
excluding the link to apply for the teleconsultation from corresponding CRM content when the teleconsultation is not permitted, when transmitting each piece of CRM content.

10. The computer-implemented method of claim 9, wherein the link to apply for the teleconsultation is provided by being included in corresponding CRM content when a timepoint at which each piece of CRM content is transmitted is in a predetermined period of time from an immediately previous consultation of the recipient.

11. A non-transitory computer-readable storage medium storing instructions for performing the computer-implemented method of claim 1.

12. An electronic device comprising:
one or more processors;
a communication interface operatively coupled to the one or more processors and configured to provide communication with a medical institution client and a terminal of a recipient; and
a storage configured to store piece of customer relationship management (CRM) content,
wherein the one or more processors are configured to perform the computer-implemented method of claim 1.
